(19) United States
(12) Patent Application Publication
(10) Pub. No.: US 2017/0129970 A1
(43) Pub. Date: May 11, 2017

(54) IMPROVED POPPET FOR CRYOGENIC FLUID COUPLING

(71) Applicant: JC CARTER LLC, Richmond Heights, OH (US)

(72) Inventors: Richard N. Henfling, Richmond Heights, OH (US); Mikhail Demin, Mayfield Heights, OH (US)

(73) Assignee: JC CARTER LLC, Richmond Heights, OH (US)

(21) Appl. No.: 15/123,957

(22) PCT Filed: Mar. 5, 2015
(86) PCT No.: PCT/US15/18982

§ 371 Date: Sep. 6, 2016

Related U.S. Application Data
(60) Provisional application No. 61/948,821, filed on Mar. 6, 2014.

(51) Int. Cl. C08F 8/40; C08F 8/42; C08F 20/36; C08F 20/60; A61K 51/06; C08G 73/02

(52) U.S. Cl. CPC C08F 8/40; A61K 51/06; C08G 73/0206; C08F 20/36; C08F 20/60; C08F 8/42

(57) ABSTRACT

Subject polymers have a plurality of repeating units having amino-bis-phosphonate functionality pendent groups of a polyacrylate, polymethacrylate, polyacrylamide, polymethacrylamide, or linear polyethyleneimine. The polymers are prepared from a plurality of monomers comprising amino-te-phosphonate functionality or from a polymer having NHBoc or active ester pendent groups that are subsequently transformed into the amino-bis-phosphonate comprising polymer. The amino-bis-phosphonate comprising polymers or their precursor polymers can be prepared by RAFT polymerization or by transformation of a preformed polymer. The amino-bis-phosphonate comprising polymers can be linear or branched polymers. The amino-bis-phosphonate comprising polymers can be complexed with metal ions. The metal ions can be a radionuclide and the amino-bis-phosphonate comprising polymer complexes can be used as radiotherapeutic agents.

### CROSS-REFERENCE TO A RELATED APPLICATIONS

This application claims the benefit of U.S. Provisional Application Ser. No. 61/952,681, filed Mar. 13, 2014, and U.S. Provisional Application Ser. No. 62/081,049, filed Nov. 18, 2014, the disclosures of which are hereby incorporated by reference in its entireties, including all figures, and drawings.

This invention was made with government support under DMR-1203146 awarded by the National Science Foundation. The government has certain rights in the invention.

### BACKGROUND OF INVENTION

Phosphorous containing materials have garnered increasing attention in recent years due to their varied potential uses, particularly for flame retardancy. Uses include biomedical applications, fuel cell membranes, corrosion inhibiting agents, and metal chelators. Among the materials investigated are phosphorus-containing polymers. Bis-phosphonates and amino-bis-phosphonates, or nitrogen-containing bis-phosphonates (NBPs), have emerged as a class of phosphorous compounds with applications in metal chelation and in medicine. Vinyl addition polymers containing amino-bis-phosphonate ester moieties are disclosed in, for example, Chougrani et al., European Polymer Journal, 2008, 44 1771-81 and Martin et al. FR 2892420.

One application target of amino-phosphonates has been for radiopharmaceuticals. For example, the preparation of [^{117m}Sn]Sn(IV)-N,N′,N′-trimethylenephosphonate-poly-(ethyleneimine), D. R. Jansen et al., Journal of Inorganic Biochemistry, 2009, 103 1265-72, from N,N′,N′-trimethylenephosphonate-polyethyleneiminepolyethyleneimine PEI-MP has been examined and found lacking as a bone-seeking radiopharmaceutical. PEI-MP is prepared from relatively poorly defined branched polyethyleneimine which contains primary, secondary and tertiary amines groups, and forms amino-phosphonate as well as amino-bis-phosphonates units along the polymer. Hence the failure of the PEI-MP based radiopharmaceutical possibly results from insufficiencies inherent to the PEI-MP.

The main disadvantages in using polyethyleneimine (PEI) is that its highly branched structure raised issue with regard to both binding and control over size. However, its high water solubility was a decisive advantage, one that was often noticeably lacking in several generations of new materials that included an all-carbon polymer backbone. Solubility issues, as well as the ever-present possibility of amide hydrolysis, caused us to consider alternatives, which ironically led back to PEI-based materials, albeit with a crucial difference. Linear PEI (LPEI) can be synthesized by cationic polymerization of 2-oxazolines and subsequent hydrolysis of the resulting polyamide (FIG. 1), and polyoxazolines themselves have been used in biological contexts. Polymers based on this backbone would then combine the necessary molecular weight control with the vital water-solubility.

An effective radionuclide therapy, particularly for osteosarcoma (bone tumors), with a chelated radionuclides, such as Sm, remains a goal. To this end, bis-phosphonates remain desirable as bis-phosphonates target bone. Radical polymerization has been carried out to prepare phosphate-, phosphonate-, amino-bis-phosphonate-, and phosphonic acid-containing polymers. Reversible addition fragmentation chain transfer (RAFT) polymerization has been used to synthesize polymers containing phosphate-, phosphonate-, and phosphonic acid-containing polymers. RAFT polymerization permits control of radical polymerization systems, including molecular weight control and the formation of branched polymers without gelation as is common with common radical polymerization protocols. Such features hold promise for optimizing tumor targeting of phosphonate ligated radionuclides. Additionally, the synthesis of LPEI-based materials that incorporated EDTMP-like ligands is desirable to produce a polymer with high water solubility, controlled molecular weight, and favorable binding capabilities.

### BRIEF DESCRIPTION OF DRAWINGS

FIG. 1 shows a chain polymerization of 2-oxazoline and subsequent conversion to LPEI.

FIG. 2 is a reaction scheme for the preparation of linear NHBoc polymers by a RAFT polymerization, according to an embodiment of the invention.

FIG. 3 shows the synthesis of LPEI-MP.

FIG. 4 shows the synthesis of linear polyethyleneimine-ethylenediamine tetra(methylene phosphonic acid) LPEI-EDTMP.

FIG. 5 is a reaction scheme for the preparation of amino-bis-phosphonate polymers by transformation of the linear NHBoc polymers by a RAFT polymerization, according to an embodiment of the invention.

FIG. 6 is a reaction scheme for the preparation of branched NHBoc polymers by a RAFT polymerization employing polymerizable CTAs, according to an embodiment of the invention.

FIG. 7 shows gel permeation chromatography (GPC) traces of linear NHBoc polymers prepared by RAFT polymerization, according to an embodiment of the invention.

FIG. 8 is a reaction scheme for the preparation of branched NHBoc polymers by a RAFT copolymerization employing diacrylate or dimethacrylate comonomers, according to an embodiment of the invention.

FIG. 9 illustrates the lack of selectivity of small molecule radionuclide therapeutic agents as opposed to polymeric radionuclide therapeutic agents, according to an embodiment of the invention.

FIG. 10 shows the complexation of Sm ion by a pair of coupled amino-bis-phosphonate, which is a model for the complexation that forms with the amino-bis-phosphonate polymers, according to an embodiment of the invention.

FIG. 11 shows the plot of the hydrodynamic volume of various amino-bis-phosphonate polymers, according to embodiments of the invention, and polyethyleneimine based aminophosphonate polymers.

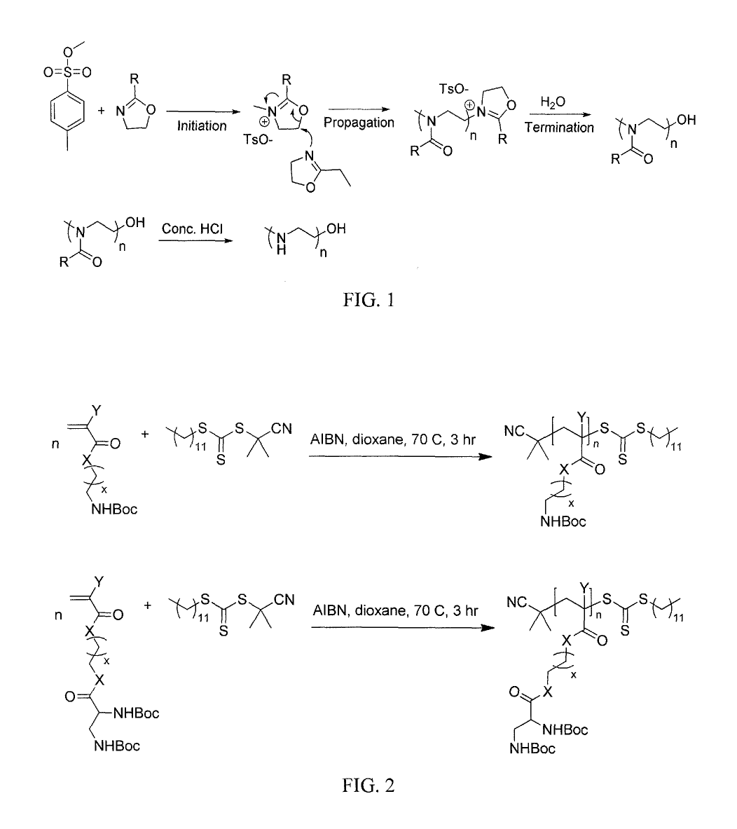

### DETAILED DISCLOSURE

Embodiments of the invention are directed to the controlled polymerization and copolymerization of acrylate, methacrylate, acrylamide, and methacrylamide comprising monomers and to the homopolymers and copolymers therefrom that further comprise amino-bis-phosphonate side groups. As used herein, the term polymer is indicative of a homopolymer and/or a copolymer, as one skilled in the art can readily appreciate situations that the polymer is necessarily a homopolymers or a copolymer. As used herein, a homopolymerization can ultimately yield a copolymer by subsequent reactive modification of the homopolymers formed, as can be appreciated by those of ordinary skill in the art, and the term copolymer does not infer the nature of the polymerization process with respect to the composition of monomers employed. As used herein, polymerization can be indicative of the polymerization of one or more different monomers. In embodiments of the invention, a portion of the monomers contain side groups that include or provide reactive functionality for the formation of side groups with at least two amino-bis-phosphonates groups in a homopolymer or copolymer. In embodiments of the invention, a branched copolymer is prepared, where the branched site results from a comonomer. Copolymers can possess two or more different repeating units. In embodiments of the invention, the molecular weight and molecular weight distribution is controlled, primarily to achieve a desired hydrodynamic radius of the amino-bis-phosphonate comprising polymers and copolymers in aqueous solution. The hydrodynamic radius can be chosen to promote selective permeability of the homopolymers and copolymers into tumor cells over normal healthy cells. In embodiments of the invention, the homopolymers and copolymers are free of amino-mono-phosphonate groups. In embodiments of the invention, the homopolymers and copolymers include complexes with radionuclides for use in radionuclide therapies.

Embodiments of the invention are directed to the preparation of polymers by reversible addition fragmentation chain transfer (RAFT) polymerization. RAFT polymerization permits the control of coupling, disproportionation, and transfer process in a radical polymerization. The RAFT radical of the chain end is in equilibrium as a stable radical adduct and the propagating polymer chain radical such that, generally, the equilibrium is dominantly populated by the adduct and not the polymer radical. In this manner, the degree of polymerization is more readily controlled over a normal free radical polymerization and the processes that can result in gelation can be suppressed when preparing a branched copolymer. In embodiments of the invention, monomers that contain the RAFT functionality as a pendent group to an acrylate, methacrylate, acrylamide, and methacrylamide can be the initiation site of a branch of the copolymer when used with monomers that ultimately provide the amino-bis-phosphonate functionality.

In an embodiment of the invention, NHBoc monomers are of the structure:

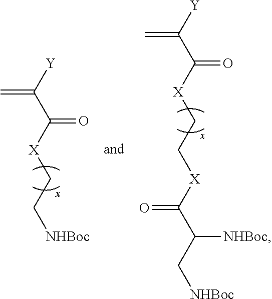

where Y is H or CH₃, X is independently NH or O, and x is 1 to 10. These monomers can be used separately or in any mixture thereof to prepare polymers thereof. The monomers can be used with other acrylate, methacrylate, acrylamide, or methacrylamide monomers having any groups attached to the polymerizable olefin functionality. In an embodiment of the invention, the polymerization is via a RAFT polymerization. These monomers can be polymerized in a non-aqueous solution, for example, in dioxane, tetrahydrofuran (THF), or any other organic solvent. In embodiments of the invention, homopolymers or copolymers are prepared with one or more of the repeating units:

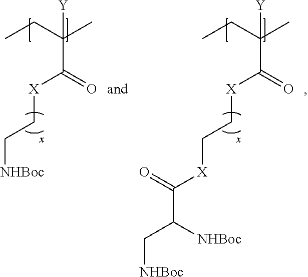

where Y, X, and x are as defined for the monomers that provide the repeating units. The polymerization can be carried out to any degree of polymerization, from 2 to 1,000 or more. Copolymerization can be carried out with other acrylate, methacrylate, acrylamide, and/or methacrylamide monomers.

These polymers can undergo substitution reaction to form poly(amino-bis-phosphonates) of the structure:

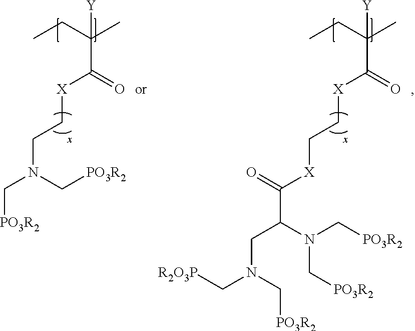

where Y is H or CH₃, X is independently NH or O, x is 1 to 10, and R is independently H, methyl, ethyl, C₃ to C₁₄ alkyl, C₆ to C₁₄ aryl, C₇ to C₁₄ alkylaryl, C₇ to C₁₄ arylalkyl, or any of these hydrocarbon groups with one or more hydroxyl, halo, C₁ to C₁₄ alkoxy, C₆ to C₁₄ aryloxy, C₇ to C₁₄ arylalkyloxy, C₇ to C₁₄ alkylaryloxy, C₆ to C₁₄ aryloxyamino, cyano, alkylcarboxy, arylcarboxy, C₂-C₁₄ dialkylamino, the hydrogen phosphonate and/or phosphonate salt, where the cation of the salt is any alkali metal, alkali earth metal, any ammonium ion, or other metal where the metal ion can be complexed to one or more mono-, bi-, or polydentate ligands. The formation of the amino-bis-phosphonate groups can be to some or all of the NHBoc functionality of the precursor polymer. During the transformation of the NHBoc functionality, hydrolysis can occur to various degrees at the NHBoc functionality, to form amines, or at the C(O)X functionality, to form amine, alcohol, or carboxylic acid functionality on an amino-bis-phosphonate copolymer.

In an embodiment of the invention, amino-bis-phosphonate monomers are prepared of the structure:

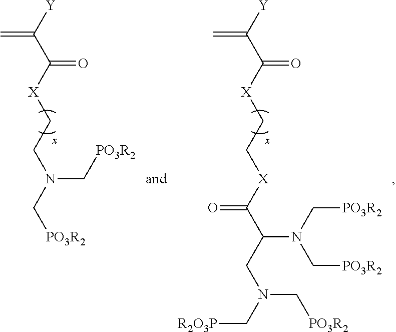

where Y is H or CH₃, X is independently NH or O, x is 1 to 10, and R is H, methyl, ethyl, C₃ to C₁₄ alkyl, C₆ to C₁₄ aryl, C₇ to C₁₄ alkylaryl, C₇ to C₁₄ arylalkyl, or any of these hydrocarbon groups with one or more hydroxyl, halo, C₁ to C₁₄ alkoxy, C₆ to C₁₄ aryloxy, C₇ to C₁₄ arylalkyloxy, C₇ to C₁₄ alkylaryloxy, C₆ to C₁₄ aryloxyamino, cyano, alkylcarboxy, arylcarboxy, C₂-C₁₄ dialkylamino, any alkali metal ion, any alkali earth metal ion, any ammonium ion, transition metal ion, lanthanide metal ion, or actinide metal ion, where the metal ion is optionally complexed with one or more mono-, bi-, or polydentate ligands. The amino-bis-phosphonate monomers can be polymerized using the RAFT method in aqueous or non-aqueous solution to form poly(amino-bis-phosphonates) homopolymers or copolymers.

These monomers can be prepared from the NHBoc monomers of equivalent Y, X, and x, or they can be prepared by any other method. The monomers can be prepared from active esters of acrylic acid or methacrylic acid. For example, the acrylate or methacrylate of pentafluorophenol can be converted to the amino-bis-phosphonate monomers by reaction with the nucleophiles or nucleophiles derived from:

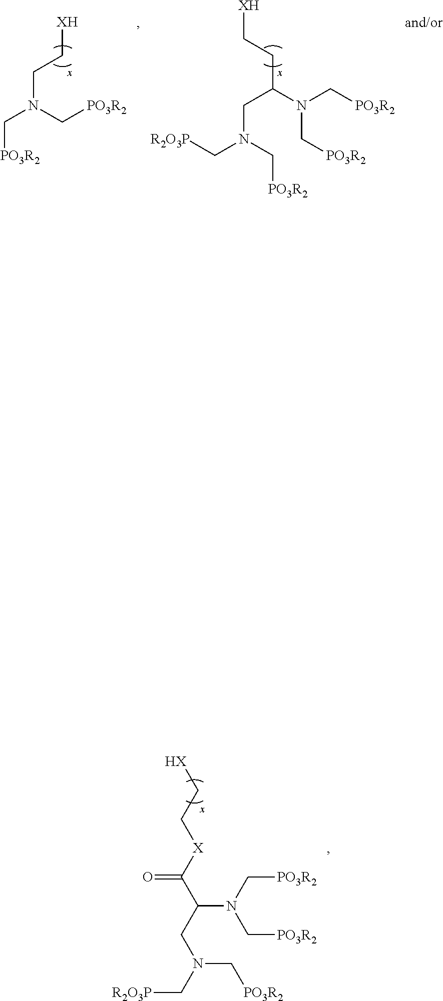

where X is independently NH or O, x is 1 to 10, and R is independently H, methyl, ethyl, C₃ to C₁₄ alkyl, C₆ to C₁₄ aryl, C₇ to C₁₄ alkylaryl, C₇ to C₁₄ arylalkyl, or any of these hydrocarbon groups with one or more hydroxyl, halo, C₁ to C₁₄ alkoxy, C₆ to C₁₄ aryloxy, C₇ to C₁₄ arylalkyloxy, C₇ to C₁₄ alkylaryloxy, C₆ to C₁₄ aryloxyamino, cyano, alkylcarboxy, arylcarboxy, or C₂-C₁₄ dialkylamino. Alternately, these nucleophiles can be used for direct substitution on homopolymers or copolymers of active esters of acrylic acid or methacrylic acid to form poly(amino-bis-phosphonate) homopolymers and copolymers. For example, by direct nucleophilic substitution on a homopolymer or copolymer having repeating units of the structure:

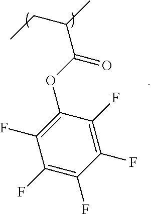

In an embodiment of the invention, the RAFT adduct is provided by using an alkylsulfanylthiocarbonyl-sulfanyl-substituted alkane chain transfer agent (CTA) of the structure:

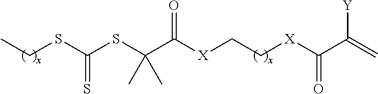

where Y is H or CH₃, X is independently NH or O, and x is independently 0 to 11, which provides the stabilized radical adduct, such as, for example, 2-dodecylsulfanylthiocarbonyl-sulfanyl-2-methylpropionic acid and a radical initiator, such as, for example, 2,2′-azobisisobutyronitrile (AIBN) for polymerizations carried out in non-aqueous solution and, for example, 2,2′-azobis(2-methylproprionamidine dihydrochloride) for polymerization carried out in aqueous solution. In embodiments of the invention, the CTA can be an alkylsulfanylthiocarbonyl-sulfanyl-substituted alkane where the substituent terminates with an acrylate, methacrylate, acrylamide, or methacrylamide polymerizable group to provide branching sites by polymerization of the CTA. Exemplary branching CTAs that can be use are 2-((2-(((Dodecylthio)carbonothioyl)thio)-2-methylpropanoyl)oxy)ethyl acrylate and 2-((2-(((Dodecylthio)carbonothioyl)thio)-2-methylpropanoyl)oxy)ethyl acrylamide:

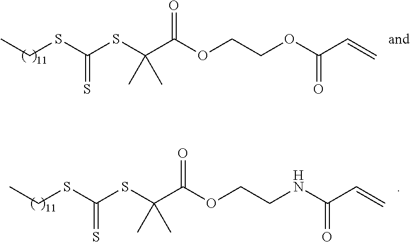

In an embodiment of the invention, a bis-acrylate, bis-methacrylate, bis-acrylamide or bis-methacrylamide of the structure:

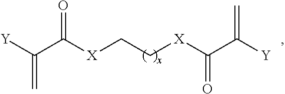

where Y is H or CH₃, X is independently NH or O, and x is 0 to 11 provides branching sites to a polymer formed by RAFT polymerization.

In an embodiment of the invention, RAFT polymerization is carried out with an NHBoc functional monomer to form a homopolymer as indicated in FIG. 2. The homopolymer will be linear with a degree of polymerization that is dependent upon the ratio of monomers to CTAs and initiator. In an exemplary embodiment, as shown in FIG. 2, the proportions of the NHBoc monomer, N-tert-butyloxycarbonyl-N′-acryl-1,2-diaminoethane, CTA, and AIBN were varied as indicated in Table 1, below.

TABLE 1
Polymerization of various molar ratios of N-tert-butyloxycarbonyl-
N′-acryl-1,2-diaminoethane to CTA and initiator.

| monomer | CTA | AIBN | Mn (theoretical) | Mn (GPC) | PDI |
|---|---|---|---|---|---|
| 78 | 1 | 0.1 | 15,000 | 15,600 | 1.43 |
| 130 | 1 | 0.1 | 25,000 | 22,200 | 1.53 |
| 182 | 1 | 0.1 | 35,000 | 25,000 | 1.56 |
| 233 | 1 | 0.1 | 45,000 | 33,300 | 1.39 |

The superiority of the RAFT polymerization to prepare the linear polymer was apparent in comparison to polymerizations lacking the RAFT CTA. As can be seen in Tables 2 and 3, below, the molecular weight reflected the monomer CTA ratio whereas the monomer to initiator ratio of more traditional radical polymerizations displayed poor control of the molecular weight and gave broader molecular weight distributions.

Another embodiment of the invention is directed to a water-soluble polymer having ethylene imine (EI) repeating units, LPEI-EDTMP of the structure:

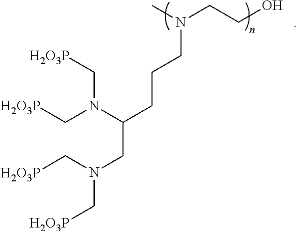

This linear polymer has the advantages of narrow molecular weight distribution, water-solubility, and good binding capabilities. In addition to an exemplary LPEI-EDTMP that has been synthesized and evaluated in vivo, there are many other possibilities for polymers based on a LPEI backbone, for example copolymers with unsubstituted ethyleneimine or C₁-C₁₆ alkyl substituted ethylene imine repeating units. The LPEI-EDTMP can be used to chelate radionuclides for treatment of cancer and other disorders using a solubilized and stable delivery system, according to an embodiment of the invention.

In an embodiment of the invention, polymerization of a ligand-bearing 2-oxazoline can form a LPEI-EDTMP or equivalent thereof. This approach is an alternative to polymerization followed by a post-polymerization functionalization steps.

In other embodiments of the invention, other ligands beyond EDTMP could be installed and evaluated. These materials may have uses in addition to radionuclide delivery. For example, LEPI-MP could potentially be investigated as a new water-solubilizing group and used in ways analogous to PEG.

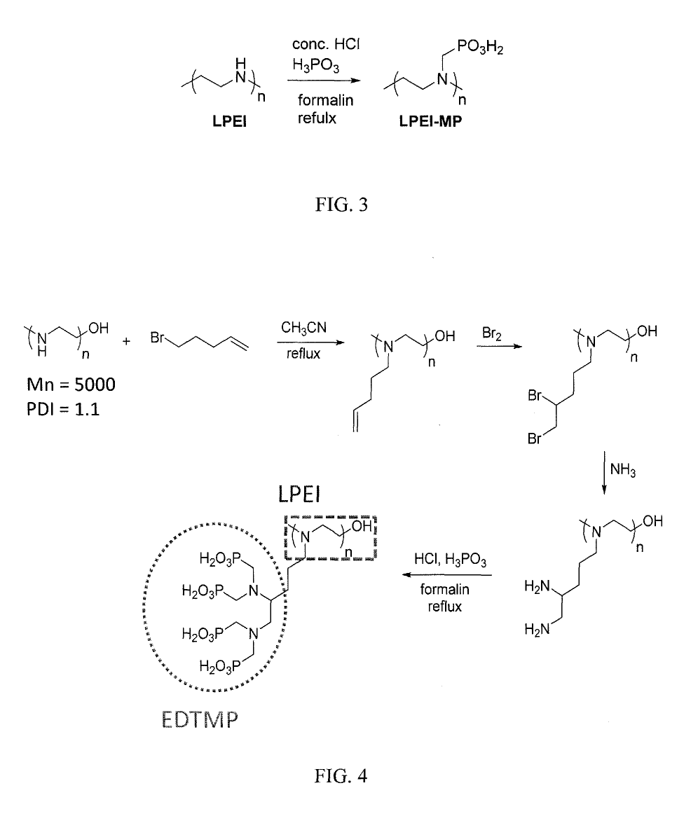

Because of the commercial availability of linear polyethyleneimine (LPEI) at a variety of molecular weights, post-polymerization modification can be carried out as an alternate to preparing from a parent 2-oxazoline. The synthesis of LPEI-MP is shown in FIG. 3. However, this material displayed poorer binding abilities than PEI-MP.

Alkylation of LPEI has the benefit of resulting in a polymer without readily hydrolysable groups, whereas poly(oxazoline)s have an amide moiety in the repeat unit. The use of acetonitrile, as shown in FIG. 4, or DMF as solvent with an organic base is possible, however, using potassium carbonate in ethanol results in a clean product and clear characterization. As the reaction progresses, potassium bromide salt precipitated from the reaction solution, which drives the reaction to completion. The salt can be removed by filtration and the product isolated by evaporating the remaining solvent.

The alkylated product is brominated and converted to the amine by addition of aqueous ammonium hydroxide. The intermediate dibromoalkyl product is not soluble in water, however, as the reaction progresses, the polymer became water soluble. The resulting ethylenediamine-bearing polymer is isolated as the hydrobromide salt. This polymer is readily water-soluble, unlike the alkylated parent compound, and is converted to the desired LPEI-EDTMP.

Binding studies with LPEI-EDTMP revealed this polymer binds well, as determined by dialysis experiments. Consequently, this material was tested in vivo in a small mouse study (n=8). Structurally, this material has several advantages. Its backbone is water soluble and it lacks hydrolysable groups.

A drug kit to test this new material in mice requires that: the concentration of Sm is sufficiently high so as to be detectable by ICP-MS such that a significant portion of the dose is deposited in a given organ; the injectable volume has to be tolerable for a 20 g mouse (approximately 100-200 μL); and the dose and polymer ligand-to-metal ratio is comparable to that used with ¹⁵³Sm-EDTMP (Quadramet™). The exact composition of Quadramet™, however, is not precise, and calculations assume the maximum amount of Sm provided in a Quadramet label.

A dose of 0.02 μg Sm (with associated polymer ligand) was used per mouse, which roughly corresponds to the amount of Sm in a typical Quadramet™ dose, though the absolute amount is much smaller given that it is for a 20 g mouse. As with Quadramet™, the formulation contains a large excess of ligand to ensure that all Sm is bound. No signs of toxicity were observed in the mice after administration of Sm-LPEI-EDTMP solution as injected into the tail vein). No mice died prior to being sacrificed three hours after administration.

TABLE 2
Comparison of RAFT and traditional radical polymerization
of N-tert-butyloxycarbonyl-N′-acryl-1,2-diaminoethane

| Conditions | Mn (kDa) | PDI |
|---|---|---|
| AIBN (1000:1, M:I) , benzene, 65° C., 4 h | 86 | 3.7 |
| AIBN (1000:1, M:I), dioxane (w/inhibitor), 65° C., | 20 | 2.7 |
| 22 h | | |
| RAFT (131:1:0.05 M:CTA:I), dioxane (w/inhibitor), | 10 | 2.1 |
| 70° C., 21 h | | |
| RAFT (262:1:0.2, M:CTA:I), DMA, 70° C., 24 h | 19 | 2.0 |
| RAFT (262:1:0.5, M:CTA:I), DMA, 70° C., 24 h | 21 | 1.9 |
| AIBN (1000:1, M:I), DMA, 70° C., 24 h | 53 | 2.6 |

TABLE 3
Comparison of RAFT and traditional radical polymerization of

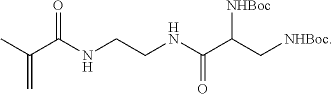 [SMILES: C=C(C)C(=O)NCCNC(=O)C(CNC(=O)OC(C)(C)C)NC(=O)OC(C)(C)C]

| Conditions | Mn (kDa) | PDI |
|---|---|---|
| AIBN (1000:1, M:I), dioxane (w/inhibitor), 65 oC, 22 h | | |
| AIBN (1000:1, M:I), DMF, 65° C., 14 h | 19 | 2.4 |
| RAFT (73:1:0.05 M:CTA:I), DMA, 70° C., 19 h | 6 | 2.0 |
| RAFT (146:1:0.2, M:CTA:I), DMA, 70° C., 48 h | 17 | 2.8 |
| RAFT (262:1:0.5, M:CTA:I), DMA, 70° C., 24 h | 18 | 2.1 |
| AIBN (1000:1, M:I), DMA, 70° C., 24 h | 39 | 3.1 |

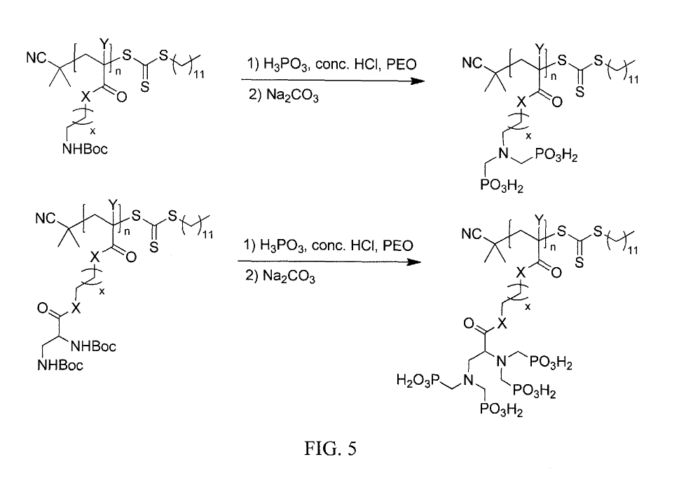

As shown in FIG. 5, the resulting NHBoc polymer can be transformed into the amino-bis-phosphonate polymer. Although, illustrated for complete conversion of NHBoc to amino-bis-phosphonate, side reactions result in copolymers where hydrolysis products are also formed on the pendant groups of the polymer. Pendent groups formed by hydrolysis side reactions of an NHBoc methacrylamide, for example, from N-tert-butyloxycarbonyl-N′-acryl-1,2-diaminoethane, include:

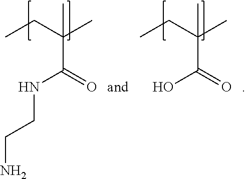

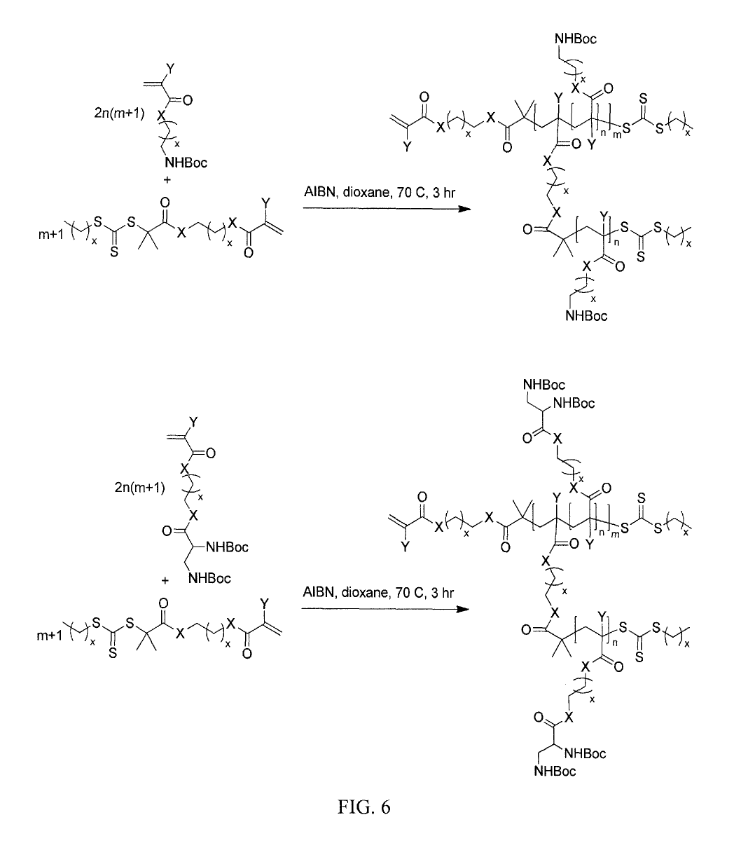

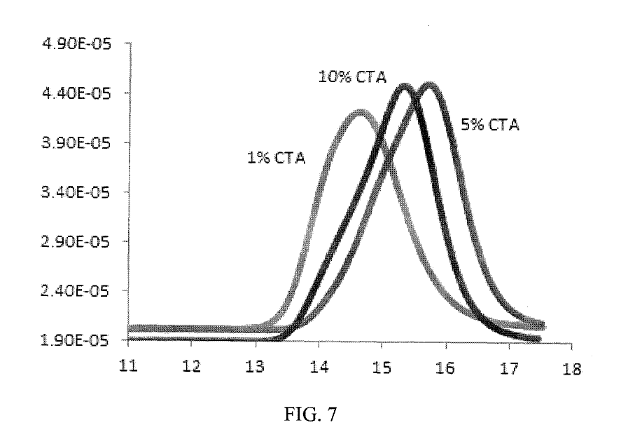

In an embodiment of the invention, the RAFT polymerization uses an alkylsulfanylthiocarbonyl-sulfanyl-substituted alkane chain transfer agent (CTA) with an NHBoc monomer to form a branched polymer, as illustrated in FIG. 6. Using 2-((2-(((Dodecylthio)carbonothioyl)thio)-2-methylpropanoyl)oxy)ethyl acrylate and N-tert-butyloxycarbonyl-N′-acryl-1,2-diaminoethane, the branching of the polymers are obvious from comparison of their molecular weight with the theoretical molecular weight assuming that the CTA only acted as a chain transfer agent and did not participate as a comonomer in Table 4, below. The GPC traces of the polymers are shown in FIG. 7. In the manner illustrated in FIG. 6 for the linear NHBoc polymer, the branched NHBoc polymers can be transformed into branched amino-bis-phosphonate polymers.

TABLE 4
Polymerization of various molar ratios of N-tert-butyloxycarbonyl-
N′-acryl-1,2-diaminoethane to CTA.

| | | MW mono | | | |
|---|---|---|---|---|---|
| Monomer | CTA | (theoretical) | Mn | Mw | PDI |
| 100 | 1 | 21,400 | 39,400 | 51,700 | 1.32 |
| 100 | 1 | 21,400 | 36,400 | 49,700 | 1.36 |
| 20 | 1 | 4,290 | 13,400 | 17,600 | 1.32 |
| 20 | 1 | 4,290 | 10,300 | 14,800 | 1.44 |
| 10 | 1 | 2,140 | 14,800 | 20,200 | 1.37 |

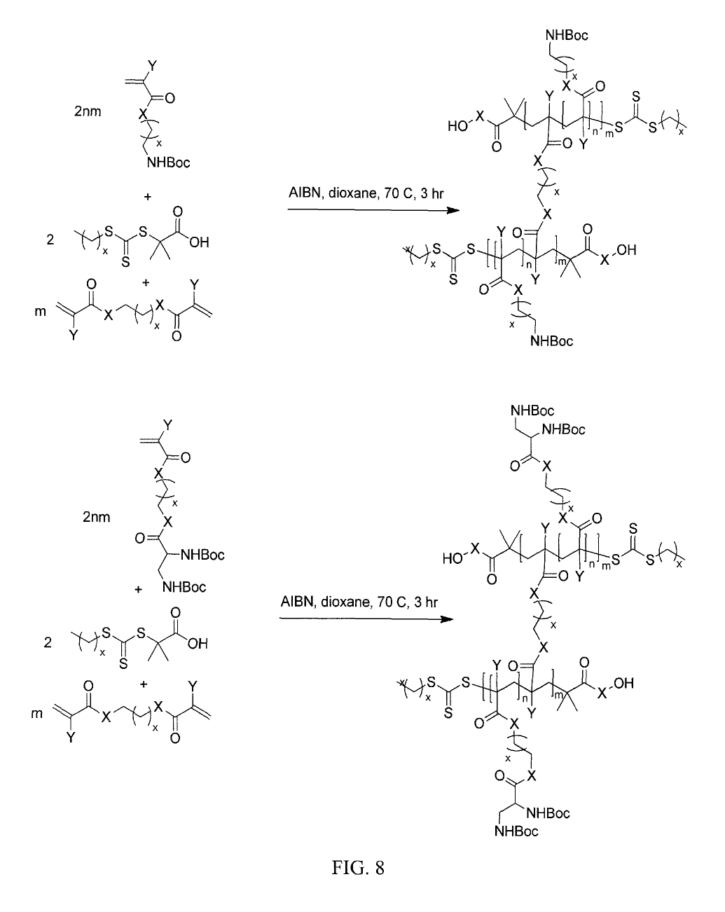

In an embodiment of the invention, the branched polymer is prepared by the use of a non-polymerizable CTA and a difunctional monomer, as shown in FIG. 8. Using 2-((2-(((Dodecylthio)carbonothioyl)thio)-2-methylpropanoic acid, N-tert-butyloxycarbonyl-N′-acryl-1,2-diaminoethane, and bis-acrylamidomethane the branching is obvious from the comparison of the molecular weight calculated for the bis-acrylamide reacting at only one end in Table 5, below. In the manner illustrated in FIG. 6 for the linear NHBoc polymer, the branched NHBoc polymers can be transformed into branched amino-bis-phosphonate polymers.

TABLE 5
Polymerization of various molar ratios of N-tert-butyloxycarbonyl-
N′-acryl-1,2-diaminoethane, bis-acrylamidomethane, and CTA.

| | | | MW | | | |
|---|---|---|---|---|---|---|
| | Cross- | | monoreacted | | | |
| | linker | | CL | | | |
| monomer | (CL) | CTA | (theoretical) | Mn | Mw | PDI |
| 100 | 1 | 1 | 21,400 | 24,240 | 33,950 | 1.4 |
| 200 | 10 | 1 | 45,900 | 37,290 | 55,990 | 1.501 |
| 200 | 20 | 1 | 45,900 | 133,000 | 190,100 | 1.429 |
| 200 | 40 | 1 | 45,900 | 187,600 | 551,700 | 2.94 |

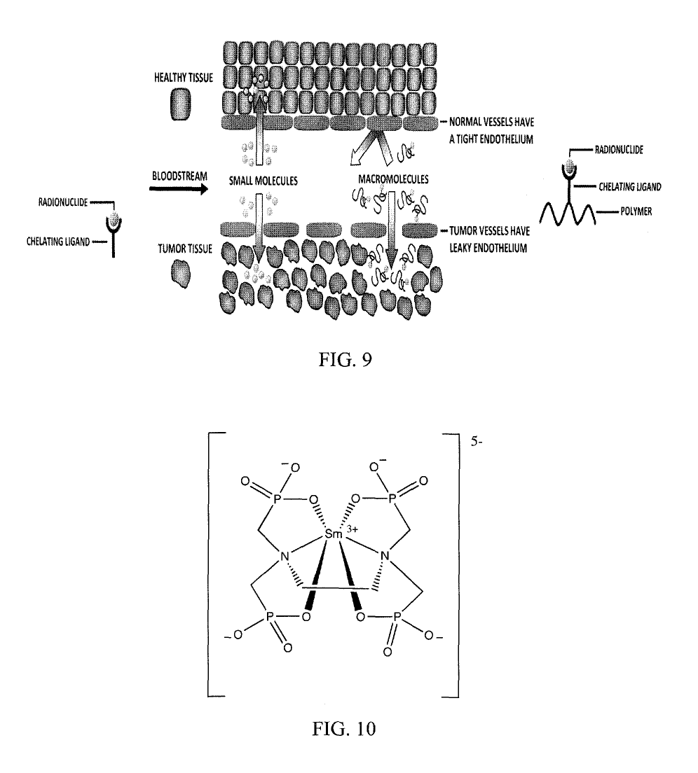

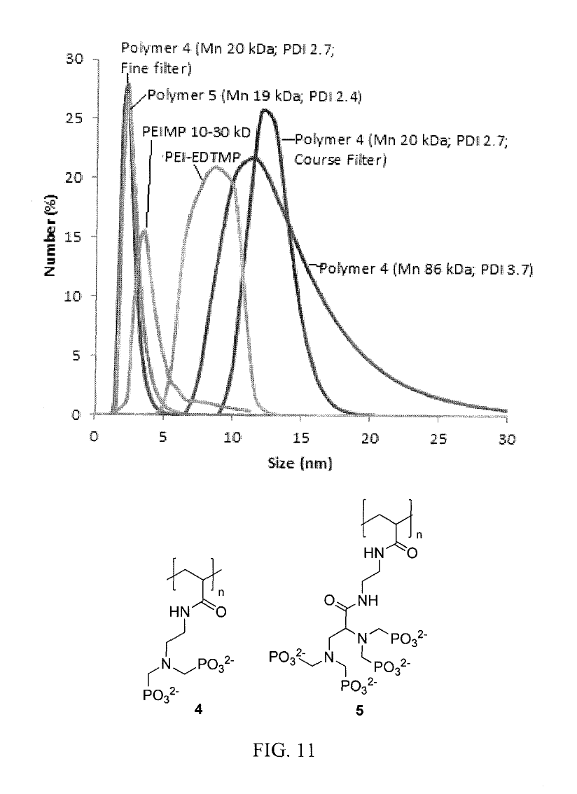

In embodiments of the invention, the polymers are prepared to have a desired hydrodynamic radius for a desired use. In an embodiment of the invention the polymer is used for a delivery vehicle of radionuclides to tumor cells selectively over healthy cells. This is shown in FIG. 9, where the advantage of a polymeric delivery vehicle to cancer cells. In an embodiment of the invention amino-bis-phosphonates, as shown in FIG. 10 for a pair of amino-bis-phosphonates separated by an ethylene unit complex ¹⁵³Sm. In the same manner, the amino-bis-phosphonate polymers of FIG. 5 can complex ¹⁵³Sm in this manner. Other metals that can be complexed include, but are not limited to, Tc, Eu, Gd, Fe, and U. FIG. 11 shows dynamic light scattering plots of various aminophosphonate polymers, where polymers can be prepared that are of hydrodynamic diameters that is in agreement of the size range of glomerular pores, which is 5 to 15 nm.

In embodiments of the invention, the amino-bis-phosphonate polymers can be formed as copolymers of other monomers, including acrylic acid, methacrylic acid, acrylate terminated polyethylene oxide, methacrylate terminated polyethylene oxide, and other water soluble monomers to from water soluble polymers. Additionally, the CTA can be one that provides a water soluble pendent group, such as polyethylene oxide, for example:

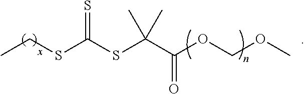

In this manner, relatively small amino-bis-phosphonate polymers by RAFT polymerization can be in the form of a block copolymer of polyethylene oxide, where an oligo(amino-bis-phosphonate) provides for nuclide binding.

In addition to polymerizations that are according to the conditions given in Tables 2 and 3 above, embodiments of the invention are further described in the examples below.

### Methods and Materials

Materials and Measurements.

All reagents were obtained from commercial sources and used as provided, with the exception of 1,4-dioxane, which was purified via basic alumina plug. Anhydrous solvents were obtained from an anhydrous solvent system and used immediately. All ¹H NMR (300 MHz) and ¹³C NMR (75 MHz) spectra were recorded on a Varian Mercury 300 spectrometer. Chemical shifts for ¹H and ¹³C NMR were referenced to residual signals from CDCl₃ (¹H 7.25 ppm and ¹³C 77.00 ppm). Gel permeation chromatography (GPC) was performed at 40° C. using a Waters Associates GPCV2000 liquid chromatography system with an internal differential refractive index detector and two Waters Styragel HR-5E columns (10 μm PD, 7.8 mm i.d., 300 mm length) using HPLC grade THF as the mobile phase at a flow rate of 1.0 mL/min.

### Synthesis of tert-butyl-N-(2-aminoethyl)carbonate

A solution of 1,2-diaminoethane (161.46 g, 2.686 mol, 9 equiv.) was prepared in 300 mL of 1,4-dioxane and stirred. A second solution of di-tert-butyldicarbonate (65.60 g, 0.298 mol, 1 equiv.) in 400 mL of 1,4-dioxane was added to the first over a 2 h period. The reaction was stirred for 22 hours. 1,4-dioxane was evaporated and the solution was precipitated in 500 mL H₂O. Insoluble bis(N,N′-t-butyloxycarbonyl)-1,2-diaminoethane was removed from the solution by filtration. The filtrate was extracted with 4 200 mL portions of CH₂Cl₂ and dried with MgSO₄. MgSO₄ was filtered off and the remaining solvent was removed by rotary evaporation. A colorless oil was obtained in 69% (32.96 g) yield.

### Synthesis of N-tert-butyloxycarbonyl-N′-acryl-1,2-diaminoethane

A 3 neck round bottom flask was placed under argon with an attached addition funnel. Tert-butyl-N-(2-aminoethyl)carbonate (32.96 g, 0.206 mol, 1 equiv.), 100 mL of triethylamine, and 350 mL of anhydrous CH₂Cl₂ were added to the flask. The flask was cooled to −20 degrees C. with an ice-salt bath. 200 mL of anhydrous CH₂Cl₂ and acryloyl chloride (185 mL, 0.227 mol, 1.1 equiv.) were transferred to the addition funnel, added drop wise over a 2 h period, and left stirring for 16 h. The solution was extracted with 4 250 mL portions of H₂O and the organic layer was dried with MgSO₄, CH₂Cl₂ was removed by rotary evaporation. A solid white product was obtained in 86.14% (38.02 g) yield. The product was dissolved in ethyl acetate and run through a silica plug for further purification.

### Synthesis of Poly(N-tert-butyloxycarbonyl-N′-acryl-1,2-diaminoethane)

N-tert-butyloxycarbonyl-N′-acryl-1,2-diaminoethane was added to a vials with a 1,3,5-trioxane internal standard. Stock solutions of CTA and AIBN in 1,4-dioxane were prepared. AIBN stock solution, CTA stock solution, and additional 1,4-dioxane were added to the vials to achieve a concentration of 1.5 M. Vials were purged with argon for 20 min, then placed in an silicon oil bath until they reached approximately 90% conversion, as determined by NMR and internal standard. After removal, additional 1,4-dioxane was added to the vials and the solutions were precipitated into cold hexanes. A fluffy white solid was obtained after the solution was filtered.

### Functionalization of Poly(N-tert-butyloxycarbonyl-N′-acryl-1,2-diaminoethane)

Two synthesis methods were pursued for installing methyl phosphate groups. In the first route, H₃PO₃ (1.29 g, 9.32 mmol, 4 equiv.), poly(N-tert-butyloxycarbonyl-N′-acryl-1,2-diaminoethane) (0.5 g, 2.33 mmol, 1 equiv.), and paraformaldehyde (0.35 g, 11.65 mmol, 5 equiv.) were added to a flask and placed in a silicon oil bath at 90 degrees C. Excess conc. HCl was added and a reflux condenser was attached. The reaction was left to stir for 24 h. The reaction was neutralized with saturated NaHCO₃ solution. The solution was evaporated by rotary evaporation to give a brown solid.

In the second route, phosphonate esters were installed first and subsequently de-protected. Poly(N-tert-butyloxycarbonyl-N′-acryl-1,2-diaminoethane) (0.5 g, 2.33 mmol, 1 equiv.), diethyl phosphite (0.71 g, 5.13 mmol, 2.2 equiv.), and paraformaldehyde (0.15 g, 5.13 mmol, 2.2 equiv.) were added to a flask with 200 mL of THF. A cloudy solution resulted. The flask was placed in a silicon oil bath at 80 degrees C. with a condenser and chiller. The reaction was left to stir for 24 h. After 24 h, the solution was clear and was evaporated by rotary evaporation. The flask with the isolated compound was purged with argon and 30 mL of anhydrous CH₂Cl₂ was added. The flask was placed in an ice water bath and TMSBr (1.6 mL, 12.1 mmol, 10 equiv.) was dripped in via syringe. Reaction was left to stir for 24 h. The solvent was then removed by rotary evaporation and a solid product was obtained. The product was dissolved in 200 mL of MeOH and left to stir for 24 h. Solvent was removed by rotary evaporation to give a yellow-orange solid.

### Poly(alkylated ethyleneimines)

A representative synthesis of an alkylated LPEI is given. Linear PEI was alkylated by nucleophilic substitution with 5-bromopentene. Linear PEI (0.5 g, 1 eq.) was dissolved in approximately 50 mL of ethanol. To this solution, 2.75 mL (2 eq.) of 5-bromopentene and 6.43 (4 eq.) of potassium carbonate were added. The solution was brought to reflux, but even at this temperature the potassium carbonate was not fully soluble. After 24 hours the reaction was cooled to room temperature. White precipitate (presumably potassium bromide) was observed. The salt was filtered off and rinsed with dichloromethane. The combined ethanol/dichloromethane solutions were then rotavapped to give 0.76 g of product as a cloudy, viscous oil.

¹H NMR (300 MHz, CDCl₃): δ=5.77 (CH₂═CH—CH₂—), 4.943 (CH₂—CH—), 2.484 (—NCH₂CH₂N—), 2.414 (t, —NCH₂CH₂CH₂CH═CH₂), 2.01 (dt, —CH₂CH═CH₂), 1.496 (tt, —NCH₂CH₂CH₂CH═CH₂)

¹³C NMR (300 MHz, CDCl₃): δ=138.52 (CH₂═CH—CH₂—), 114.63 (CH₂═CH—), 54.61, 53.15, 31.57, 26.48.

Linear PEI-MP

Linear PEI and H₃PO₃ were combined in a round bottom flask and solvated with 20 mL of water. 20 mL of concentrated HCl was added and the flask was put in a silicon oil bath at reflux. The reagents were not completely dissolved. Formalin was added to the flask and after approximately an hour, the solution was pink and the components were still not dissolved. The reaction was left overnight and was precipitated in ethanol.

Sm-LPEI-EDTMP Kit Preparation for Mouse Studies

In this study, a non-radioactive isotope of Sm was used. Due to the very low concentrations required, stock solutions were made. First, 4.3 mg SmCl₃.6H₂O was added to a vial and dissolved in 19.99 g of endotoxin-free water (concentration=0.215 mg/mL). From this solution, 12.4 μL was transferred to a new vial, which ultimately held the final solution. To this new vial 3.23 mg of LEPI-EDTMP was added as well as 15.2 mg of sodium bicarbonate and 20.02 g water. This final solution was then sealed in the vial and heated in an oil bath at 70° C. for 2 minutes after which it was cooled to room temperature. The anticipated dose is then 0.02 μg Sm (150 μL solution) per mouse.

All publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A amino-bis-phosphonate polymer, comprising a plurality of repeating units of at least one of: 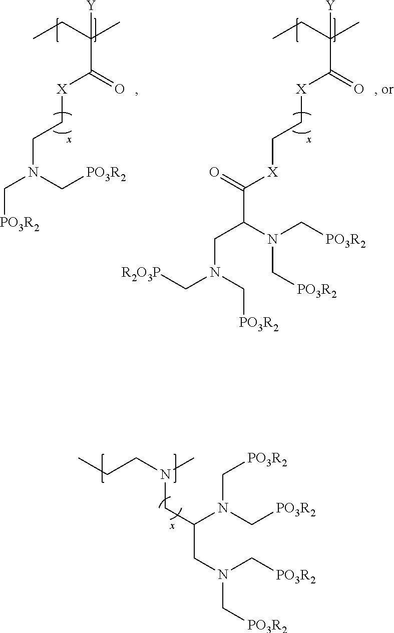
where Y is H or CH₃, X is independently NH or O, x is 1 to 10, and R is independently H, methyl, ethyl, C₃ to C₁₄ alkyl, C₆ to C₁₄ aryl, C₇ to C₁₄ alkylaryl, C₇ to C₁₄ arylalkyl, or any of these hydrocarbon groups with one or more hydroxyl, halo, C₁ to C₁₄ alkoxy, C₆ to C₁₄ aryloxy, C₇ to C₁₄ arylalkyloxy, C₇ to C₁₄ alkylaryloxy, C₆ to C₁₄ aryloxyamino, cyano, alkylcarboxy, arylcarboxy, C₂-C₁₄ dialkylamino, any alkali metal ion, any alkali earth metal ion, any ammonium ion, transition metal ion, lanthanide metal ion, or actinide metal ion, where the metal ion is optionally complexed with one or more mono-, bi-, or polydentate ligands.

2. The amino-bis-phosphonate polymer according to claim 1, wherein the repeating unit is an acrylate or methacrylate, further comprising a plurality of end-groups of the structure: 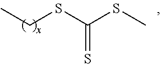
where x is 1 to 11.

3. The amino-bis-phosphonate polymer according to claim 1, wherein the repeating unit is an acrylate or methacrylate, further comprising repeating units from water soluble acrylates and/or methacrylates.

4. The amino-bis-phosphonate polymer according to claim 1, wherein the repeating unit is an acrylate or methacrylate, further comprising branching repeating units and terminal groups derived from chain transfer agents of the structure: 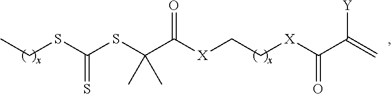
where Y is H or CH₃, X is independently NH or O, and x is independently 0 to 11.

5. The amino-bis-phosphonate polymer according to claim 1, further comprising branching repeating units from difunctional monomers of the structure: 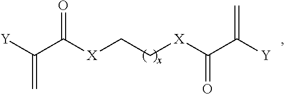
where Y is H or CH₃, X is independently NH or O, and x is 0 to 11.

6. A monomer for the preparation of the amino-bis-phosphonate polymer according to claim 1, comprising: 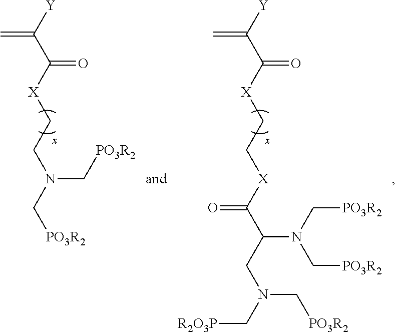
where Y is H or CH₃, X is independently NH or O, x is 1 to 10, and R is H, methyl, ethyl, C₃ to C₁₄ alkyl, C₆ to C₁₄ aryl, C₇ to C₁₄ alkylaryl, C₇ to C₁₄ arylalkyl, or any of these hydrocarbon groups with one or more hydroxyl, halo, C₁ to C₁₄ alkoxy, C₆ to C₁₄ aryloxy, C₇ to C₁₄ arylalkyloxy, C₇ to C₁₄ alkylaryloxy, C₆ to C₁₄ aryloxyamino, cyano, alkylcarboxy, arylcarboxy, C₂-C₁₄ dialkylamino, any alkali metal ion, any alkali earth metal ion, any ammonium ion, transition metal ion, lanthanide metal ion, or actinide metal ion, where the metal ion is optionally complexed with one or more mono-, bi-, or polydentate ligands.

7. The amino-bis-phosphonate polymer according to claim 1, wherein the repeating unit is an EI-EDTMP metal chelate, comprising: 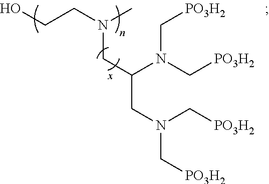
and the transition metal ion, lanthanide metal ion, or actinide metal ion, wherein the transition metal ion, lanthanide metal ion, or actinide metal is a portion of a radionuclide salt chelated to one or more of the repeating units.

8. The amino-bis-phosphonate polymer according to claim 7, further comprising repeating units of the structure: 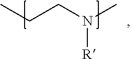
wherein R′ is H or C₁-C₁₆ alkyl.

9. A polymer for the preparation of the amino-bis-phosphonate polymer according to claim 1, comprising repeating units of the structure: 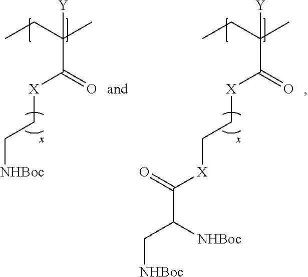
where Y is H or CH₃, X is independently NH or O, x is 1 to 10, and R is independently H, methyl, ethyl, C₃ to C₁₄ alkyl, C₆ to C₁₄ aryl, C₇ to C₁₄ alkylaryl, C₇ to C₁₄ arylalkyl, or any of these hydrocarbon groups with one or more hydroxyl, halo, C₁ to C₁₄ alkoxy, C₆ to C₁₄ aryloxy, C₇ to C₁₄ arylalkyloxy, C₇ to C₁₄ alkylaryloxy, C₆ to C₁₄ aryloxyamino, cyano, alkylcarboxy, arylcarboxy, C₂-C₁₄ dialkylamino, any alkali metal ion, any alkali earth metal ion, any ammonium ion, transition metal ion, lanthanide metal ion, or actinide metal ion, where the metal ion is optionally complexed with one or more mono-, bi-, or polydentate ligands.

10. The polymer according to claim 9, further comprising a plurality of end-groups of the structure: 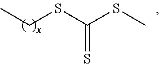
where x is 1 to 11.

11. The polymer according to claim 9, further comprising repeating units from water soluble acrylates and/or methacrylates.

12. The polymer according to claim 9, further comprising branching repeating units and terminal groups derived from chain transfer agents of the structure: 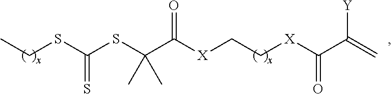
where Y is H or CH₃, X is independently NH or O, and x is independently 0 to 11.

13. The polymer according to claim 9, further comprising branching repeating units from difunctional monomers of the structure: 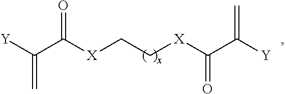
where Y is H or CH₃, X is independently NH or O, and x is 0 to 11.

14. A method for the preparation of the polymer according to claim 9, comprising:
providing a plurality of monomers wherein at least one of the monomers is of the structure: 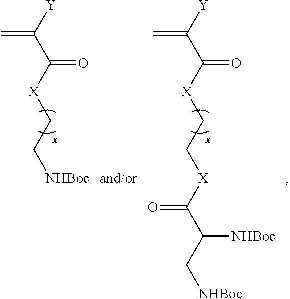
where Y is H or CH₃, X is independently NH or O, and x is 1 to 10;
providing an alkylsulfanylthiocarbonyl-sulfanyl-substituted alkane chain transfer agent (CTA);
mixing the CTA with the plurality of monomers and a radical initiator in a solvent to form a polymerization mixture; and
heating the polymerization mixture, wherein the amino-bis-phosphonate polymer is formed.

15. The method according to claim 14, wherein the alkylsulfanylthiocarbonyl-sulfanyl-substituted alkane chain transfer agent (CTA) is of the structure: 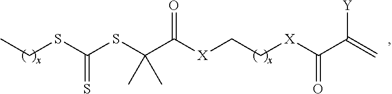
where Y is H or CH₃, X is independently NH or O, and x is independently 0 to 11.

16. The method according to claim 14, further comprising providing a plurality of difunctional monomers of the structure: 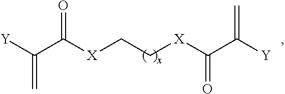
where Y is H or CH₃, X is independently NH or O, and x is 0 to 11, and adding the difunctional monomers to the polymerization mixture.

17. A method for the preparation of the amino-bis-phosphonate polymer according to claim 1, comprising:
providing a plurality of monomers wherein at least one of the monomers is of the structure: 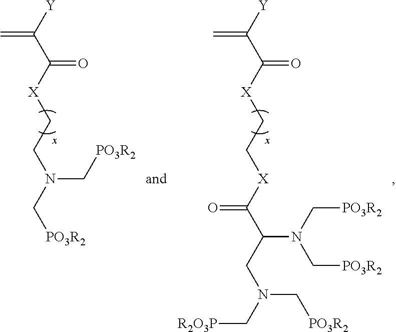
where Y is H or CH₃, X is independently NH or O, x is 1 to 10, and R is H, methyl, ethyl, C₃ to C₁₄ alkyl, C₆ to C₁₄ aryl, C₇ to C₁₄ alkylaryl, C₇ to C₁₄ arylalkyl, or any of these hydrocarbon groups with one or more hydroxyl, halo, C₁ to C₁₄ alkoxy, C₆ to C₁₄ aryloxy, C₇ to C₁₄ arylalkyloxy, C₇ to C₁₄ alkylaryloxy, C₆ to C₁₄ aryloxyamino, cyano, alkylcarboxy, arylcarboxy, C₂-C₁₄ dialkylamino, any alkali metal ion, any alkali earth metal ion, any ammonium ion, transition metal ion, lanthanide metal ion, or actinide metal ion, where the metal ion is optionally complexed with one or more mono-, bi-, or polydentate ligands;
providing an alkylsulfanylthiocarbonyl-sulfanyl-substituted alkane chain transfer agent (CTA);
mixing the CTA with the plurality of monomers and a radical initiator in a solvent to form a polymerization mixture; and
heating the polymerization mixture, wherein the amino-bis-phosphonate polymer is formed.

18. The method according to claim 17, wherein the alkylsulfanylthiocarbonyl-sulfanyl-substituted alkane chain transfer agent (CTA) is of the structure: 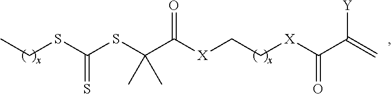
where Y is H or CH₃, X is independently NH or O, and x is independently 0 to 11.

19. The method according to claim 17, further comprising providing a plurality of difunctional monomers of the structure: 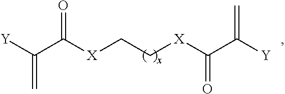
where Y is H or CH₃, X is independently NH or O, and x is 0 to 11, and adding the difunctional monomers to the polymerization mixture.

20. A method for the preparation of the amino-bis-phosphonate polymer according to claim 1, comprising:
providing an active ester polymer with a plurality of repeating units of the structure: 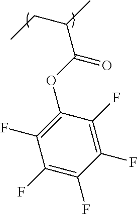
or other active ester repeating units;
providing a nucleophilic reagent or a precursor to a nucleophilic reagent of the structure: 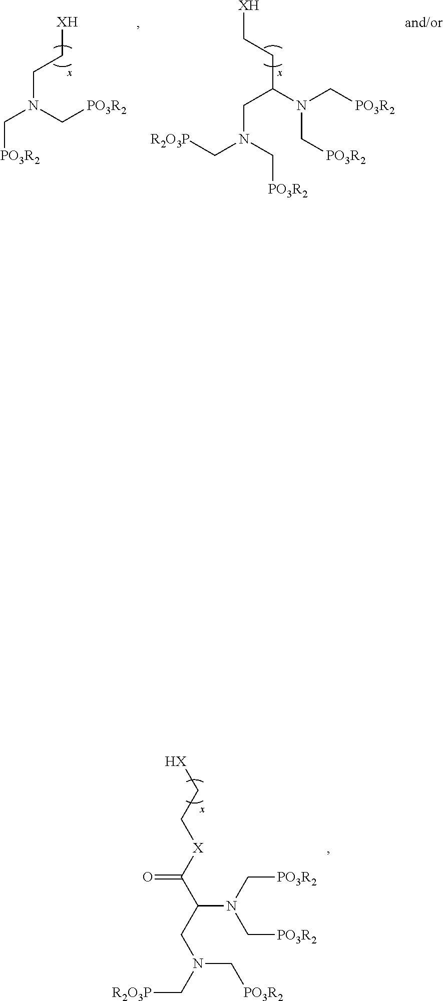
where X is independently NH or O, x is 1 to 10, and R is independently H, methyl, ethyl, C₃ to C₁₄ alkyl, C₆ to C₁₄ aryl, C₇ to C₁₄ alkylaryl, C₇ to C₁₄ arylalkyl, or any of these hydrocarbon groups with one or more hydroxyl, halo, C₁ to C₁₄ alkoxy, C₆ to C₁₄ aryloxy, C₇ to C₁₄ arylalkyloxy, C₇ to C₁₄ alkylaryloxy, C₆ to C₁₄ aryloxyamino, cyano, alkylcarboxy, arylcarboxy, or C₂-C₁₄ dialkylamino;
mixing the active ester polymer with the nucleophilic reagent or the precursor to a nucleophilic reagent in a solvent to form a reaction mixture; and
heating the reaction mixture, wherein the amino-bis-phosphonate polymer according to claim 1 is formed.

21. A method for the preparation of the amino-bis-phosphonate polymer according to claim 1, comprising:
providing the polymer according to claim 9;
mixing the polymer with phosphoric acid or its equivalent, polyethylene oxide or its equivalent, and hydrochloric acid or its equivalent; and
neutralize at least a portion of the acid, wherein the amino-bis-phosphonate polymer according to claim 1 is formed.

22. A radiotherapeutic agent, comprising the amino-bis-phosphonate polymer according to claim 1 and at least one radionuclide complexed to the amino-bis-phosphonate polymer.

23. The radiotherapeutic agent of claim 22, wherein the radionuclide is ¹⁵³Sm.

24. A method for radiotherapy of a patient, comprising providing the radiotherapeutic agent of claim 23 to a human or animal patient.

25. A method for the preparation of the amino-bis-phosphonate polymer according to claim 1 comprising:
providing a LPEI;
providing an alpha alkenyl omega halide;
condensing the LPEI with the alpha alkenyl omega halide to form an alkenylated LPEI;
halogenating the alkenylated LPEI to form a dihaloalkyl LPEI;
condensing the dihaloalkyl LPEI with ammonia to form a diaminoalky PEI; and
adding formalin and phosphoric acid to the diaminoalky PEI to form a linear polyethyleneimine-ethylenediamine tetra(methylene phosphonic acid) LPEI-EDTMP.

26. A method for the preparation of the amino-bis-phosphonate polymer according to claim 7, comprising:
providing a linear polyethyleneimine-ethylenediamine tetra(methylene phosphonic acid) LPEI-EDTMP according to claim 25; and
combining the LPEI-EDTMP with a salt of a radionuclide.
